(19) United States
(12) Patent Application Publication
(10) Pub. No.: US 2011/0319747 A1
(43) Pub. Date: Dec. 29, 2011

(54) MAGNETIC RESONANCE DEVICE FOR USE IN A MAGNETIC RESONANCE-GUIDED ULTRASOUND TREATMENT

(76) Inventors: Sebastian Schmidt, Weisendorf (DE); Anke Weissenborn, Weil am Rhein (DE)

(21) Appl. No.: 13/167,982

(22) Filed: Jun. 24, 2011

(30) Foreign Application Priority Data
Jun. 25, 2010 (DE) 10 2010 025 060.0

(51) Int. Cl. A61B 5/055; A61N 7/00

(52) U.S. Cl. 600/411

(57) ABSTRACT

A magnetic resonance device for use in a magnetic resonance-guided ultrasound treatment has a magnet housing defining a patient receptacle and including a primary magnet, and an ultrasound device, such as an HIFU device, with a transducer unit, The magnet housing has a receptacle therein for the transducer unit of the ultrasound device, in particular in the region of the homogeneity volume.

### BACKGROUND OF THE INVENTION

1. Field of the Invention

The invention concerns a magnetic resonance device of the type for use in a magnetic resonance-guided ultrasound treatment, having a magnet housing defining a patient receptacle and including a primary magnet, and an ultrasound device, in particular an HIFU device, with a transducer unit.

2. Description of the Prior Art

Ultrasound treatments guided by magnetic resonance techniques are used for non-invasive treatment of various illnesses (for example tumors). Magnetic resonance-guided focused ultrasound (MR guided High Intensity Focused Ultrasound—HIFU) is an example. This method is used to treat, for example, myomas of the uterus. For example, an HIFU transducer is used within the magnetic resonance system in order to treat uterine myomas.

This transducer can be focused in order to reach specific penetration depths, and it can be moved—in particular via translation and/or tilting—in order to reach different positions in the body. In this treatment method it is one of the disadvantages that the patient must lie prone on the transducer or the transducer unit. The transducer requires a certain structural height and is located in a water or oil basin for better cooling and coupling. The space available for the patient in a cylindrical magnetic resonance scanner (solenoid system) is therefore severely limited. This problem is more serious in magnetic resonance scanners known as open magnetic resonance devices in which a vertical magnetic field is generated between two pole plates, so the patient receptacle is open on three sides. In such magnetic resonance devices the distance between the pole plates is typically only approximately 40 cm, such that no ultrasound treatment is possible since the transducer and the patient cannot be arranged simultaneously in the patient receptacle.

A magnetic resonance device in which an ultrasound treatment can be conducted is known from U.S. Pat. No. 6,582,381, for example. There it is proposed to install a transducer unit (including a positioning device and the transducer positioned in a water bath) in a patient table. Additional devices are also known in which the normal patient table is replaced with a different table, or is supplemented by a table overlay. However, in such solutions the person to be treated is also situated somewhat higher than the table level in a typical magnetic resonance system.

No solution exists for open magnetic resonance devices with a vertical magnetic field in which the magnet housing frequently has a C-shape since the pole plate separation in the systems available today is too small in order to also accommodate a transducer unit in addition to the person to be treated. Although a larger pole plate separation would be feasible in principle, the cost of such a magnetic resonance device would increase significantly, or marked compromises in the imaging would be necessary (poorer image quality due to lower field strength or lower homogeneity).

### SUMMARY OF THE INVENTION

An object of the present invention is to provide a magnetic resonance device in which an ultrasound treatment is possible without or with only a very small space loss in the patient receptacle.

This object is achieved by a magnetic resonance device of the aforementioned type wherein, according to the invention, the magnet housing has a receptacle for the transducer unit of the ultrasound device, in particular in the region of the homogeneity volume of the scanner (MR data acquisition unit).

According to the invention the transducer unit (which can include a positioning device for the transducer in addition to the transducer arranged in a water basin and/or oil basin) into the magnetic resonance device below a patient table and between the coils of the magnet. This means that the transducer unit inserted into the receptacle in the patient receptacle is ultimately essentially flush with the standard surface of the patient receptacle, such that the total free space in the patient receptacle is not decreased, even during a magnetic resonance-guided ultrasound treatment. For example, far more room for the patient thus remains for the treatment of uterine myomas by means of HIFU, for example. By providing a receptacle in the magnet housing whose inner surface defines the patient receptacle, such an integration for the first time also enables a realization of ultrasound treatments in open magnetic resonance devices since then the entire intervening space between the pole plates continues to be available for the person to be treated.

The receptacle can be fashioned as a depression in the magnet housing, in particular in order to achieve an optimally flush termination with the remaining surface of the magnet housing. The transducer unit is thus then countersunk into a recess in the magnet housing, such that it does not take away any space in the patient receptacle.

The transducer unit can be removable inserted into the receptacle. If the transducer unit is not required or if it should be serviced, it can consequently be pulled out upwardly into the patient receptacle (thus the bore or the space between the pole plates) and afterward be brought outside of the magnet housing.

In general, such a receptacle (in particular a depression) can be considered as a design criterion in the design of the magnetic resonance device. This means that the layout of the various coils—in particular the primary magnet, the gradient coils, the body coil and the like—is configured in the overall design of the magnetic resonance device so that the receptacle can remain free. This also enables adaptation of the cylindrical vessel of the cooling device that is provided to cool the primary magnet.

The cooling vessel arranged around the primary magnet can have a depression therein at the location of the receptacle. The cooling device that includes the cooling vessel can be fashioned as a cooling device for cooling the coil conductors of the primary magnet locally via coolant conduits and/or heat pipes that are arranged in a vacuum cooling vessel. This means that the superconducting primary magnet is not provided in a completely filled helium bath; rather, the conductors are locally cooled by locally provided cooling conduits or heat pipes associated with the coil conductors, and the cooling conduits or heat pipes are in turn located in the evacuated vacuum cooling vessel. Such a design of a cooling device for a magnetic resonance device is known from U.S. Pat. No. 7,449,889, for example. In the context of the present invention, it offers the advantage that, because the cooling vessel is already a vacuum cooling vessel and thus free vacuum spaces exist, the design of such a cooling device can be adapted in a simple manner in order to enable the accommodation of the transducer unit. In such a design of the cooling device it is advantageous for at least one control conduit and/or at least supply conduit for the transducer unit to be directed through the cooling vessel. Since the cooling vessel is ultimately evacuated and there is no helium bath that must be traversed, here such a design is possible in a particularly simple manner. Naturally, in principle it would also be conceivable to direct control and/or supply conduits through a helium bath or the like.

It is also possible for at least one control and/or at least one supply conduit for the transducer unit to be directed along through the patient receptacle (in particular below the patient table). The conduits thus are more easily accessible and thus can be serviced more easily.

As already mentioned, the different coil systems of the magnetic resonance device can be designed ab initio so as to allow the provision of the receptacle for the transducer unit, which receptacle is in particular fashioned as a depression. For this purpose, the gradient coil system can be designed as to have a recess at the location of the receptacle. Particularly in the case of a solenoid magnetic resonance device a split gradient coil is provided. Such “non-continuous” gradient coils are known, for example as described by WO 2008/122899 A1 (Split Gradient Coil). For example, such a gradient coil that ultimately is to be divided into two halves can now also advantageously be used within the scope of the present invention.

In a further embodiment at least one part of a local coil is integrated into the transducer unit, in particular at least one conductor loop of a local coil. Then it is no longer necessary to provide an additional local coil or conductor loop below the patient since this is likewise already integrated into the transducer unit. A local coil portion or a conductor loop is then provided that is located near the treatment area and thus enables image data of higher quality, in particular with a high SNR. For example, such a conductor loop can be directed through the water basin or oil basin, around the adjustment range of the transducer.

As mentioned, in order to achieve the best possible quality of the treatment the transducer should be coupled optimally well to the surface of the person being treated. For this purpose, when the magnetic resonance device has a patient table that can be introduced into the patient receptacle, the patient table can be provided with a coupling device for coupling the transducer unit to a patient surface when the region adjacent to the transducer unit has been driven into the patient receptacle. The transducer unit arranged below the patient table is thus coupled to the patient surface via the coupling device such that no (or only slight) gaps occur in the acoustic material properties. For example, a coupling body can be inserted into or can built be in, a recess of the patient table, the coupling body being a gel cushion and/or a Mylar plate, for example. The coupling device thus includes the coupling body that is used in the patient table, such that coupling is enabled when the patient table is introduced (in particular is driven in). It can be advantageous for the patient table to be able to be lowered to produce a contact between the coupling body and the transducer unit. This means that the patient table is introduced into the patient receptacle somewhat higher so that friction effects or other contact effects cannot occur; and the patient table is then lowered by a small distance in order to produce the optimally good acoustic contact.

### BRIEF DESCRIPTION OF THE DRAWINGS

FIG. 1 schematically illustrates the basic components of a magnetic resonance device according to the invention in a first embodiment.

FIG. 2 schematically illustrates the basic components of a magnetic resonance device according to the invention in a second embodiment.

### DESCRIPTION OF THE PREFERRED EMBODIMENTS

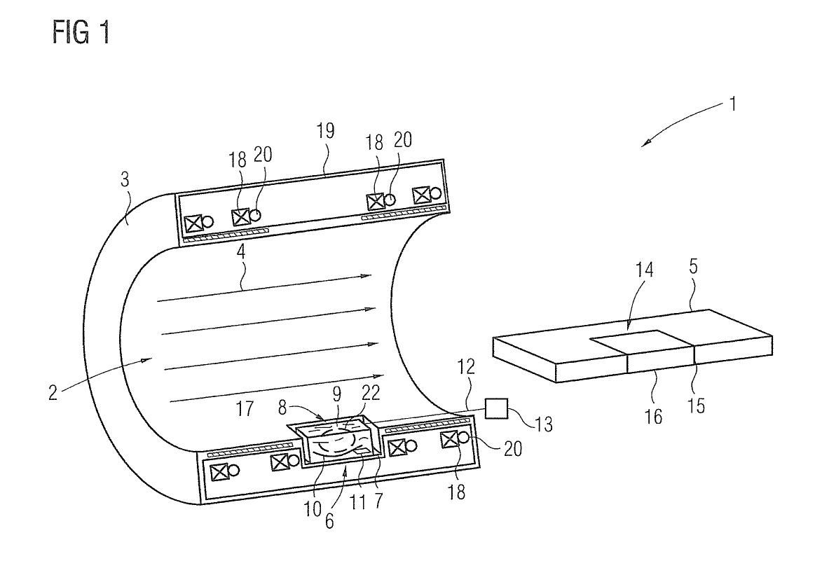

FIG. 1 shows a section view of basic components of a magnetic resonance device 1 of a first embodiment according to the invention. This is a solenoid magnetic resonance device 1 in which a magnetic field is generated in a longitudinal direction of the patient receptacle 2 that is defined by the magnet housing 3 (see arrows 4). The fundamental modes of operation and components of magnetic resonance devices are generally known, such that here only components relevant to the present invention are discussed in detail. The magnetic resonance housing 3 has a receptacle 6 (which here is fashioned as a depression 7) in a lower region that comes to lie below the patient table when the patient table 5 has been driven in. A transducer unit 8 that is part of an HIFU device is inserted into the receptacle 6. The transducer unit 8, which moreover is countersunk into depression 7 so that it can be removed, has a housing that defines a water basin 9. In the water basin 9 a transducer 10 is arranged that can be aligned and positioned variably via a positioning device 11.

In the exemplary embodiment shown here, control and supply conduits 12 are directed outward through the patient receptacle 2 below the patient table 5, for example to a control unit 13 of the HIFU device.

In order to enable a coupling of the transducer unit 8 to a patient surface, in the region that comes to lie over the transducer unit 8 when the patient table 5 has been driven in said patient table (which can be driven into the patient receptacle 2) has a coupling device 14 that presently has a coupling body inserted into a recess 15 of the patient table, here a gel cushion. So that this does not abrade the surface of the magnet housing 3 upon being driven in, the patient table 5 is driven in while being slightly raised and then can be lowered so that there is contact between the coupling body 16 and the surface of the transducer unit 8.

The depression 7 in the magnet housing 3 is achieved via a special layout of the magnetic resonance device 1. Here at least one gradient coil is initially realized as a split gradient coil (indicated at 17). This means that the gradient coil 17 has a recess in the middle that is used to realize the depression 7.

The actual basic magnet—indicated here by a few a few coil conductors 18 designated as examples—is located within a cooling vessel 19 that is part of a cooling device. The cooling device is thereby presently realized so that coolant conduits 20 are associated with the coil conductors 18, which coolant conduits 20 are directed together with the coil conduits 18 through a vacuum, which means that the cooling vessel 19 is a vacuum cooling vessel. This enables the depression 7 to be provided more simply. The primary magnet itself has already been designed so that no coil conduits 18 whatsoever are present at the location of the depression 7. It is consequently possible to design the entire magnetic resonance device 1 to that effect to enable an integration of the transducer unit 8 by means of the accommodation 6 in the magnet housing 3.

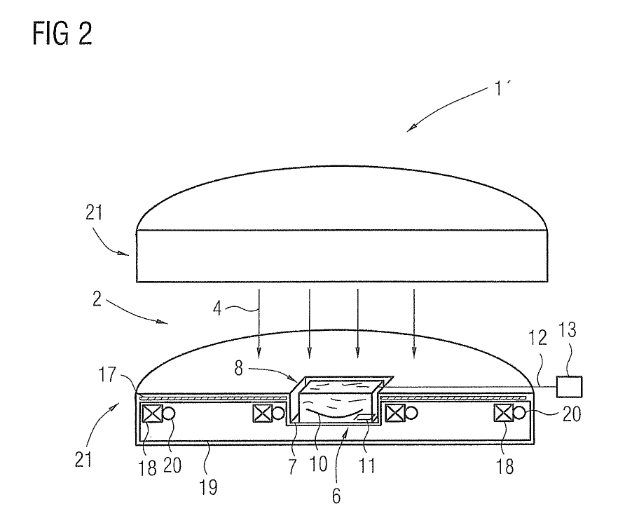

A similar realization is also possible in an open magnetic resonance device 1′, as is explained in detail via the exemplary embodiment in FIG. 2, in which (for simplicity) corresponding components are provided with the same reference characters. There only the lower of the two hollow plates 21 is drawn in section. Although no patient table 5 is shown in FIG. 2, a patient table 5 designed as in the exemplary embodiment according to FIG. 1 can be used there.

A depression 7 as a receptacle 6 for a transducer unit 8 is in turn clearly provided in the magnet housing 3. The gradient coil system 17 again has a recess. Precisely in the case of such an open magnetic resonance device 1′, an HIFU treatment is thereby advantageously possible since the patient receptacle is not or is only less limited in terms of its size.

It should be noted that the control and supply conduits 12 do not need to be directly guided through the patient receptacle 2 below the patient table. In the shown embodiments of the respective cooling devices it is also possible to direct the control and supply conduits 12 outside through the cooling vessel 19, thus ultimately through the primary magnet itself.

It can be advantageous to also integrate at least one part of a local coil—in particular a conductor loop 22 (indicated by dashes in FIG. 1)—into the transducer unit 8 since a conductor loop placed close to the patient and to the examination region is thus provided without an additional device being required.

Although modifications and changes may be suggested by those skilled in the art, it is the intention of the inventors to embody within the patent warranted hereon all changes and modifications as reasonably and properly come within the scope of his contribution to the art.

We claim as our invention:

1. A magnetic resonance apparatus comprising:
a magnetic resonance data acquisition device comprising a basic field magnet and a housing in which said basic field magnet is located, said magnet housing having a magnet housing wall that defines a receptacle adapted to receive a patient therein;
an ultrasound device adapted to treat the patient when the patient is located in the receptacle, said ultrasound device comprising a transducer unit; and
said housing wall having a receptacle therein configured to receive said transducer unit of said ultrasound device therein.

2. A magnetic resonance apparatus as claimed in claim 1 wherein said data acquisition device has a homogeneity volume in which said basic field magnet generates a magnetic field that is substantially homogenous, and wherein said receptacle is located in a region of said homogeneity volume.

3. A magnetic resonance apparatus as claimed in claim 1 wherein said receptacle for said transducer unit has a size allowing said transducer unit to fit into said receptacle without reducing a size of said patient receptacle.

4. A magnetic resonance apparatus as claimed in claim 1 wherein said transducer unit comprises a transducer and a positioning device for positioning said transducer, and a basin containing a coolant in which at least said transducer is located.

5. A magnetic resonance apparatus as claimed in claim 1 wherein said transducer unit is removably placeable in said receptacle.

6. A magnetic resonance apparatus as claimed in claim 1 wherein said receptacle is formed by a depression in said housing wall.

7. A magnetic resonance apparatus as claimed in claim 1 wherein said data acquisition device comprises a cooling vessel for said basic field magnet, and wherein said cooling vessel comprises a depression at a location of the receptacle that accommodates said receptacle in said depression.

8. A magnetic resonance apparatus as claimed in claim 7 wherein said basic field magnet comprises a coil conduit in which a coil of said basic field magnet is located, and comprising a cooling device that includes said cooling vessel, comprising coolant conductors that are located in said cooling vessel at locations in thermal communication with said coil conduit.

9. A magnetic resonance apparatus as claimed in claim 8 wherein said transducer unit comprises a conduit selected from the group consisting of a control conduit and a supply conduit, and wherein said conduit of said transducer unit is directed through said cooling vessel.

10. A magnetic resonance apparatus as claimed in claim 1 wherein said data acquisition device comprises a gradient coil system, and wherein said gradient coil system has a recess therein at a location of said receptacle for said transducer unit.

11. A magnetic resonance apparatus as claimed in claim 10 wherein said data acquisition device is a solenoid magnetic resonance device and wherein said gradient coil is a split gradient coil.

12. A magnetic resonance apparatus as claimed in claim 1 wherein said data acquisition unit comprises a radio-frequency local coil, said radio-frequency local coil comprising at least one conductor loop that is integrated into said transducer unit.

13. A magnetic resonance apparatus as claimed in claim 1 comprising a patient table movable into and out of said patient receptacle, said patient table comprising a table surface adapted to receive the patient thereon, and said patient table comprising a coupling device located in a region of said patient table that is adjacent to said transducer unit when said patient table is moved into said patient receptacle, said coupling device being configured to acoustically couple the transducer unit to the patient via said table surface.

14. A magnetic resonance apparatus as claimed in claim 13 wherein said table surface has a recess therein, and wherein said coupling device comprises a coupling body that is located in said recess.

15. A magnetic resonance apparatus as claimed in claim 14 wherein said coupling body is a gel cushion.

16. A magnetic resonance apparatus as claimed in claim 14 wherein said coupling body is a Mylar plate.

17. A magnetic resonance apparatus as claimed in claim 13 wherein said patient table comprises a table moving mechanism configured to lower said patient table in said patient receptacle to produce contact between said coupling device and said transducer unit.
